(19) United States
(12) Patent Application Publication
(10) Pub. No.: US 2007/0106129 A1
(43) Pub. Date: May 10, 2007

(54) Dietary monitoring system for comprehensive patient management

(75) Inventors: Muralidharan Srivathsa, Shoreview, MN (US); David C. Johnson, Inver Grove Heights, MN (US); Donald L. Goscha, Elk River, MN (US); Marina Brockway, Shoreview, MN (US); Veerichetty A. Kadhiresan, Centerville, MN (US)

Correspondence Address: SCHWEGMAN, LUNDBERG, WOESSNER & KLUTH/BSC-CRM, PO BOX 2938, MINNEAPOLIS, MN, 55402, US

(73) Assignee: Cardiac Pacemakers, Inc.

(21) Appl. No.: 11/269,771

(22) Filed: Nov. 7, 2005

(51) Int. Cl. A61B 5/00; G06Q 50/00

(52) U.S. Cl. 600/300; 705/3

(57) ABSTRACT

A patient management system includes a device, an interactive display, a repeater unit, and a host. The device assesses at least one dietary condition of a patient. The interactive display receives patient input related to the patient's dietary conditions and communicates dietary feedback to the patient. The system may also include at least one sensor configured to assess at least one dietary condition of the patient. The repeater unit collects information from the device, the interactive display and the sensors. The host communicates with the repeater through a network. The patient's own input of dietary information into the system can be useful for verifying sources of measured data and enhancing feedback given to the patient. The system is configured to perform accurately even in the absence of patient input or the input of incorrect information by the patient.

### TECHNICAL FIELD

The present invention generally relates to comprehensive patient management systems and, more specifically, to systems and methods for monitoring patient dietary conditions and communicating suggested dietary input for the patient.

### BACKGROUND OF THE INVENTION

Management of patients with chronic disease consumes a significant proportion of the total health care expenditure in the United States. Many of these diseases are widely prevalent and have significant annual incidences as well. Heart failure prevalence alone is estimated at over 5.5 million patients in 2000 with incidence rates of over half a million additional patients annually, resulting in a total health care burden in excess of $20 billion. Heart failure, like many other chronic diseases such as asthma, COPD, chronic pain, and epilepsy, is event driven, where acute de-compensations result in hospitalization.

Hospitalizations consume a large portion of the total health care expenditure allocated to the treatment of heart failure. Hospitalization and treatment for an acute de-compensation typically occurs after the de-compensation event has occurred. However, most heart failure patients exhibit prior non-traumatic symptoms, such as steady weight gain, in the weeks or days prior to the de-compensation. If the caregiver is aware of these symptoms, it is possible to intervene before the event, at substantially less cost to the patient and the health care system.

Intervention is usually in the form of a re-titration of the patient's drug cocktail, reinforcement of the patient's compliance with the prescribed drug regimen, or acute changes to the patient's diet and exercise. Such intervention is usually effective in preventing the de-compensation episode and thus avoiding hospitalization. Patients with chronic heart disease can receive implantable cardiac devices such as pacemakers, implantable cardioverter defibrillators (ICDs), and heart failure cardiac resynchronization therapy (CRT) devices. Currently, the electrophysiologist that implants a pacemaker or ICD requires a patient to make clinic follow up visits periodically, usually once every three or four months, in order to verify that the their implanted device is working correctly and programmed optimally. Device follow-ups are usually performed by the nurse-staff assisted by the sales representative from the device manufacturers. Device follow-ups are labor intensive and typically require patients to make multiple clinic visits.

A significant number of heart failure hospitalizations occur due to noncompliance with a strict dietary regimen. Despite adequate counseling and training during hospitalization and clinic visits, management of food intake by the patient continues to be the cause of recovery delays and even hospitalization. One primary reason for patient noncompliance with prescribed nutritional intake is the lack of adequate and timely feedback concerning the patient's dietary condition.

### SUMMARY OF THE INVENTION

The present invention generally relates to monitoring and communicating a patient's dietary conditions. The invention relates to systems and methods for measuring data associated with a patient's dietary conditions, and can include comparison of that data to an expected dietary model. Based on this comparison, one or more actions can be performed including, but not limited to, providing feedback (e.g., recommended nutritional intake, exercise, and drug therapy) to the patient related to their dietary condition, reporting to a physician, alarming, creation of new dietary models/algorithms, etc. While the patient's own input of dietary information into the system can be useful for the comparison or for verifying other sources of measured data, the system is configured to perform accurately even in the absence of patient input or the input of incorrect information by the patient.

In accordance with one aspect, the invention relates to an advanced patient management system that includes a device, an interactive display, a repeater unit, and a host. The device assesses at least one dietary condition of a patient. The interactive display receives patient input related to the patient's dietary conditions and communicates dietary feedback to the patient. The repeater unit collects information from the device and the interactive display. The device may be, for example, a patient's implanted medical device. The host communicates with the repeater through a network. The system provides dietary related feedback to the patient in response to the collected information. The system may also include at least one sensor configured to assess at least one dietary condition of the patient. Some example sensors include a sodium sensor, a weight measuring sensor, a blood pressure sensor, a heart rate sensor, an INR/Coumadin sensor, a glucose sensor, a respiration sensor, an insulin sensor, a temperature sensor, and a hydration sensor. The dietary feedback may include dietary advice, food and menu options, calorie information, sodium information, hydration conditions, pharmacological dosage recommendations, and patient input prompts.

A further aspect of the invention relates to a method of monitoring dietary conditions of a patient. The method includes providing an implanted device, a repeater, and a host in communication with each other, collecting dietary information from the patient with the implanted device, comparing the collected dietary information to expected dietary conditions of the patient stored by the repeater or the host, and communicating dietary information to the patient in response to the comparison. The method may further include providing an interactive display and collecting dietary information from the patient via the interactive display. The method may still further include providing at least one sensor configured to measure a dietary condition of the patient, and comparing the dietary condition to expected dietary conditions of the patient stored by the repeater or the host.

A still further aspect of the invention relates to a method of monitoring and communicating a patient's nutritional/dietary condition. The method includes collecting data associated with at least one dietary condition of a patient, communicating the data to a host, comparing the collected data to an expected dietary model, and communicating to the patient a recommended nutritional intake.

The above summary is not intended to describe each disclosed embodiment or every implementation of the present invention. Figures and the detailed description that follow more particularly exemplify embodiments of the invention. While certain embodiments will be illustrated and described, the invention is not limited to use in such embodiments.

### BRIEF DESCRIPTION OF THE DRAWINGS

The invention can be more completely understood in consideration of the following detailed description of various embodiments of the invention in connection with the accompanying drawings, in which:

FIG. 1 illustrates an example system made in accordance with the present invention;

FIG. 2 illustrates another example system made in accordance with the present invention;

FIG. 3 illustrates an example method for collection and analysis of a nutritional feedback system for a patient;

FIG. 4 illustrates an example advanced patient management system made in accordance with the present invention;

FIG. 5 illustrates an example interrogator/transceiver unit made in accordance with the present invention;

FIG. 6 illustrates an example interactive display for use with the example interrogator/transmitter unit of FIG. 5;

FIG. 7 illustrates an example communication system made in accordance with the present invention; and

FIG. 8 illustrates an example method for monitoring patient compliance with and efficacy of a patient drug regimen.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present invention generally relates to monitoring and communicating a patient's dietary conditions. More specifically, the present invention relates to systems and methods for measuring data associated with a patient's dietary conditions, and comparison of that data to expected dietary conditions. Based on this comparison, one or more actions can be performed including, but not limited to, providing feedback (e.g., recommended nutritional intake, exercise, and drug therapy) to the patient related to their dietary condition, reporting to a physician, issuing an alert, or creation of new dietary models/algorithms. While the patient's own input of dietary information into the system can be useful for the comparison or for verifying sources of measured data, the system is configured to perform accurately even in the absence of patient input or the input of incorrect information by the patient.

As used herein, the term “patient” is used to mean any individual from whom information is collected. The term “caregiver” is used herein to mean any provider of services, such as health care providers including, but not limited to, nurses, doctors, and other health care provider staff. The terms “dietary information” and “dietary condition” are used herein to describe any physiological condition or subjective response by a patient that relates to a patient's diet. Example physiological conditions related to a patient's diet include heart rate, glucose levels, sodium levels, electrolyte levels, blood pressure, respiration rates, temperature, and weight. Some example subjective responses by a patient related to their diet include feeling faint or dizzy, feeling thirsty, feeling bloated or gaseous, feeling nauseated, feeling heart burn, feeling weak and lethargic, and feeling tired. Other example subjective responses by a patient relate to their nutritional/dietary intake and their physical activity.

I. Monitoring and Therapy Enhancement Using Dietary Models

In the embodiments illustrated herein, nutritional/dietary monitoring and therapy for a patient can be enhanced using data collected by one or more devices associated with a patient. Specifically, one or more sensors are used to collect data associated with the dietary related conditions of the patient. Patient input related to their subjective physical conditions as well as their dietary intake and physical activity. The collected data is compared to an expected dietary model. An expected dietary model may be based upon one or more algorithms that generate different outcomes based on certain conditional inputs. The expected dietary model may be established based on, for example, the past dietary and physical history of the patient, target conditions established by a physician, industry standards and recommendations, or by patient preferences. Based on the comparison, one or more actions are performed including, but not limited to, reporting or alarming the patient, the patient's physician, or a third party, creation of new dietary model models, updating population databases, etc.

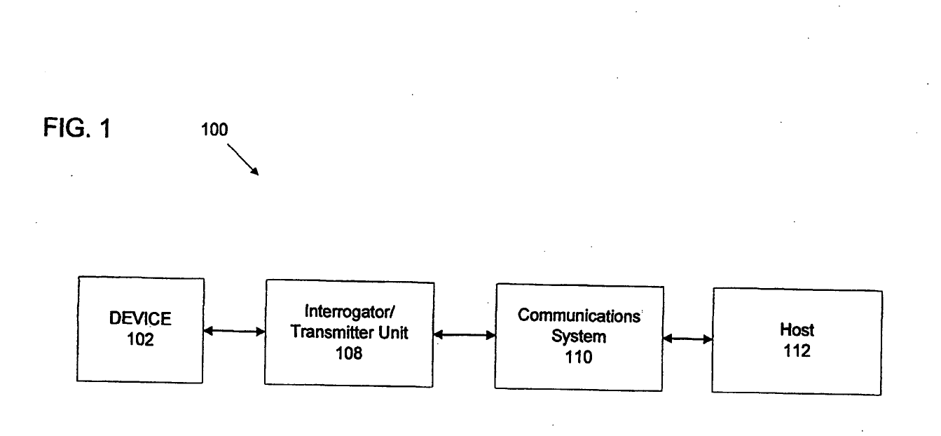

Referring now to FIG. 1, an example system 100 for collecting and analyzing patient data is illustrated. System 100 includes a device 102, an interrogatory/transceiver unit (ITU) or repeater 108, a communications system 110, and a host 112.

Device 102 is a sensor that can measure a pharmacological effect (e.g., physiological or subjective) of a drug on a patient. Device 102 can be configured in a manner similar to that of devices 104, 106, 602, 604, 606 described below. For example, device 102 can be an implanted medical device such as, for example, a cardiac rhythm management (CRM) device such as a pacemaker, a cardioverter defibrillator, or a heart failure cardiac resynchronization therapy device, or other devices such as an implantable loop recorder. Other example implanted devices include a variety of sensors such as sodium, glucose, temperature, electrolyte, hydration, respiratory, heart rate, cardiac output, and other sensors that provide information related to dietary conditions of the patient. The device 102 can also be a non-implanted device such as, for example and without limitation, a weight measuring device, a blood pressure cuff, blood content monitor (i.e., gases, glucose, creatinine, BNP), insulin sensor, or Holter or ECG devices. Device 102 can collect data associated with the patient including, but not limited to, activity level, weight, intracardiac or systemic blood pressure, heart rate, heart rate variability, thoracic impedance, or heart sounds.

Device 102 generally collects data associated with the patient and communicates the data to host 112. In one embodiment, the device 102 communicates with host 112 through ITU 108 and communications system 110. Other configurations are also possible. For example, in some embodiments, device 102 can communicate directly with communications system 110 and/or host 112. In the illustrated embodiment, device 102 can upload collected data to ITU 108 in real-time, on a periodic (batch) basis, or manually such as when interrogated by the ITU 108.

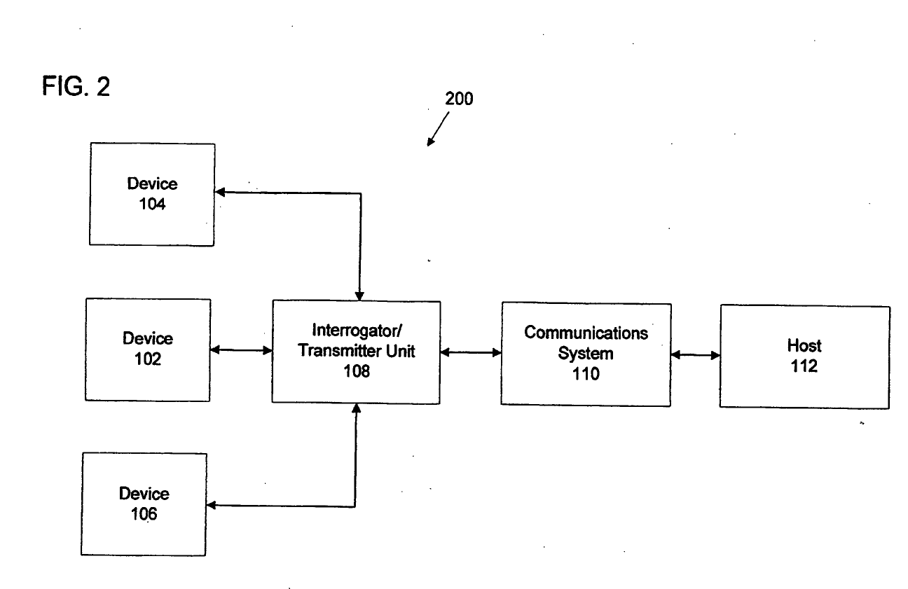

Referring now to FIG. 2, another example system 200 is illustrated. System 200 is similar to system 100 described above, except that system 200 also includes devices 104 and 106. The devices 102, 104, 106 can be any combination of implanted and external devices as described above in the discussion of sensor 102. In other embodiments, at least one of the devices 102, 104, 106 may be an interactive display or a handheld device.

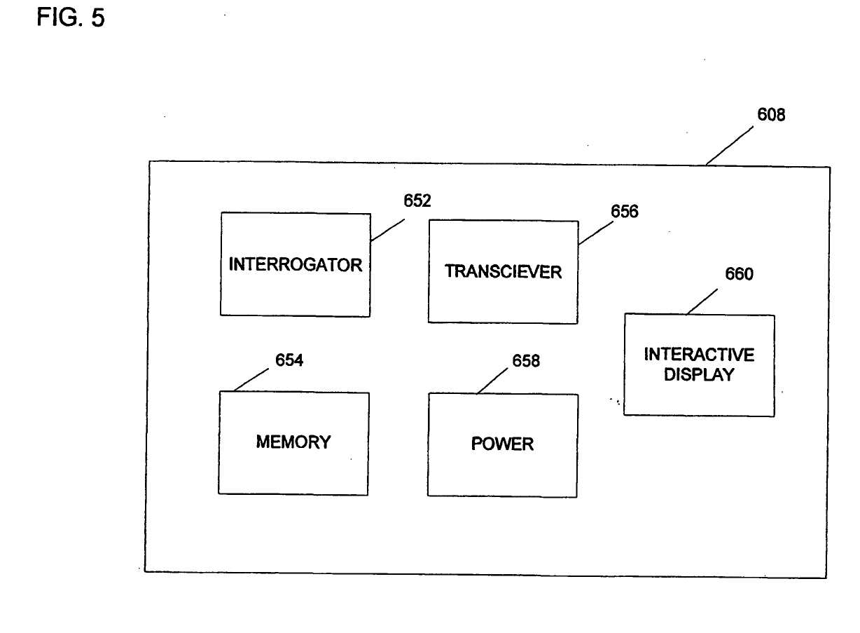

The interactive display provides for patient input to the system 200 related to, for example, nutritional/dietary intake, subjective physical conditions, and physical activity (e.g., sleep and exercise). The interactive display may also provide for communication between the system 100 and the patient using, for example, text messages related to recommended nutritional/dietary intake, physical activity, and physical conditions (e.g., glucose and sodium count). In some embodiments, the interactive display may be integrated into the ITU 108 as described below with reference to FIG. 5.

Providing one of the devices 102, 104, 106 as a handheld device could provide additional mobility and user convenience for the system 200. The ITU 108 is typically configured for use in the patient's living structure. The ITU 108 may be mobile, but is typically not convenient for carrying at all times or for use when traveling. A handheld device may be configured with some of the features of the ITU such as, for example, data collection from one of the other devices 102, 104, 106, communication with the host 112 via the communications systems 110, and analysis of some types of information, and interactive display for some patient input, dietary feedback, and sensor measurements.

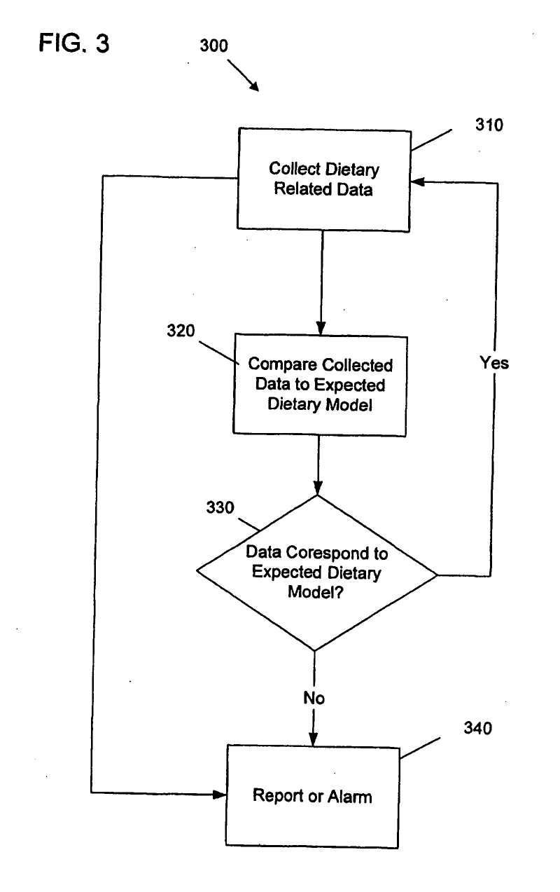

Referring now to FIG. 3, a flow diagram 300 illustrates one example method for enhancing therapy for a patient. In operation 310, data is collected about the patient using, for example, devices 102, 104, 106 (e.g., sensors, CRM device, patient input via interactive display) and information about the patient stored by the ITU 108 and host 112.

Next, in operation 320, the collected data is compared to an expected dietary model. The expected dietary model can be selected in various ways. For example, the model can be selected based on population statistics arranged, for example, according to age, race, national origin, and/or gender. In other examples, the model can be tailored according to the specific medical history of a patient. For example, an expected dietary model can be constructed based on previous data collected from the patient, dietary recommendations from a physician. Other methods for selecting an expected dietary model are also contemplated such as, for example, using individual patient genetic factors that impact a patient's blood pressure, sodium count, or cholesterol level. In yet further methods, the expected dietary model can be based upon or in some way influenced by regularly updated guidelines from such organizations as, for example, the American Hospital Association (AHA), the American Medical Association (AMA), the Heart Rhythm Society (HRS), and the Heart Failure Society of America (HFSA).

Example expected dietary models may use many parameters related to, for example, nutritional intake, physical parameters such as sodium and glucose levels, exercise, sleep, and drug regimes. A dietary model may relate to a specific parameter or to a plurality of parameters.

Referring back to FIG. 3, once the data collected from the patient is compared to the expected dietary model in operation 320, control is then passed to operation 330 and a determination is made as to whether the collected data corresponds to the expected dietary model. In one embodiment, the comparison is made by computing deviations between the models. In another embodiment, parameters of the expected model (e.g., time of the step-wise change, delta increment) are computed in advance and stored in the system.

If the collected data corresponds to the expected dietary model, control is passed back to operation 310 and collection of data continues. If the collected data does not correspond to the expected dietary model, control is passed to operation 340, and specific steps such as reporting and alarming can occur. In some embodiments, automatic steps can be initiated in response to the operation 330 such as therapy using the implanted device or altering of the patient's drug regimen.

In one example, the type and magnitude of deviation of the collected data from the expected dietary model can result in the generation various reports and alarms. In one embodiment, if the collected data indicates an adverse side effect to a given nutritional intake, a side-effect report is generated that is then forwarded to the patient via the interactive display or handheld device, and/or sent to the caregiver via, for example, the host and communications network. In yet another embodiment, if the collected data indicates noncompliance with a dietary schedule, a noncompliance report is generated that is then forwarded to the caregiver and/or patient. If the nature of the deviation from the expected dietary model is such that immediate action is desirable, alarms can be sent to the caregiver and/or to the patient to notify of the potential problem.

More than one device can also be used to measure multiple aspects of the patient's dietary condition. For example, multiple devices (or a single device with multiple capabilities) can be used to collect data related to multiple different physical effects such as, for example and without limitation, a patient's heart rate, blood pressure, temperature, sodium and glucose levels, etc. Further, in some embodiments, multiple expected dietary models can be compared to data collected with respect to the dietary condition of a patient during different activities, different times of the day or week, different dietary menus, different drug treatment, and different environmental conditions. Or, alternatively, a single expected dietary model can be used that accounts 30 for multiple conditions and inputs.

In addition, in some embodiments, not all of the collected data is forwarded to the host 112 for processing. For example, in some embodiments, the device 102 and/or the ITU 108 can conduct at least initial processing of the collected data to identify, for example, data that would indicate that immediate reporting is necessary.

Various advantages are associated with the use of systems configured in a manner similar to example systems 100 and 200 described above. For example, the collected data can be used to monitor dietary intake side effects, patient compliance, and time of nutritional intake. In addition, the data can be used to identify beneficial dietary intake in combination with certain physical activities and drug regimens. Beneficial dietary intake can be further substantiated when using the measured collect data with subjective data entered by the patient in response to system prompts for patient information.

II. Advanced Patient Management System

In some embodiments, the systems 100 and 200 described above are implemented as part of an advanced patient management (“APM”) system configured to collect patient-specific information, store and collate the information, and generate actionable recommendations to enable the predictive management of patients.

Embodiments of the APM system can be configured to monitor patient dietary conditions, determine and communicate recommended action related to the patient's dietary conditions, and develop and store a history of the patient's dietary conditions and the patient's response to the recommended action. The APM system can be configured to use dietary information and patient health history provided by at least one of a primary care giver (e.g., a doctor), a secondary caregiver database, and a population health information database in conjunction with patient physical indicators (measured or entered by the patient). The example APM systems disclosed herein can also be configured to produce reports related to a patient's current dietary conditions as well as compliance and side effects of, for example, certain dietary intake, physical activity, and drug regimens, and communicate those reports to various destinations, such as, for example, a primary caregiver, the patient, or a dietary related database.

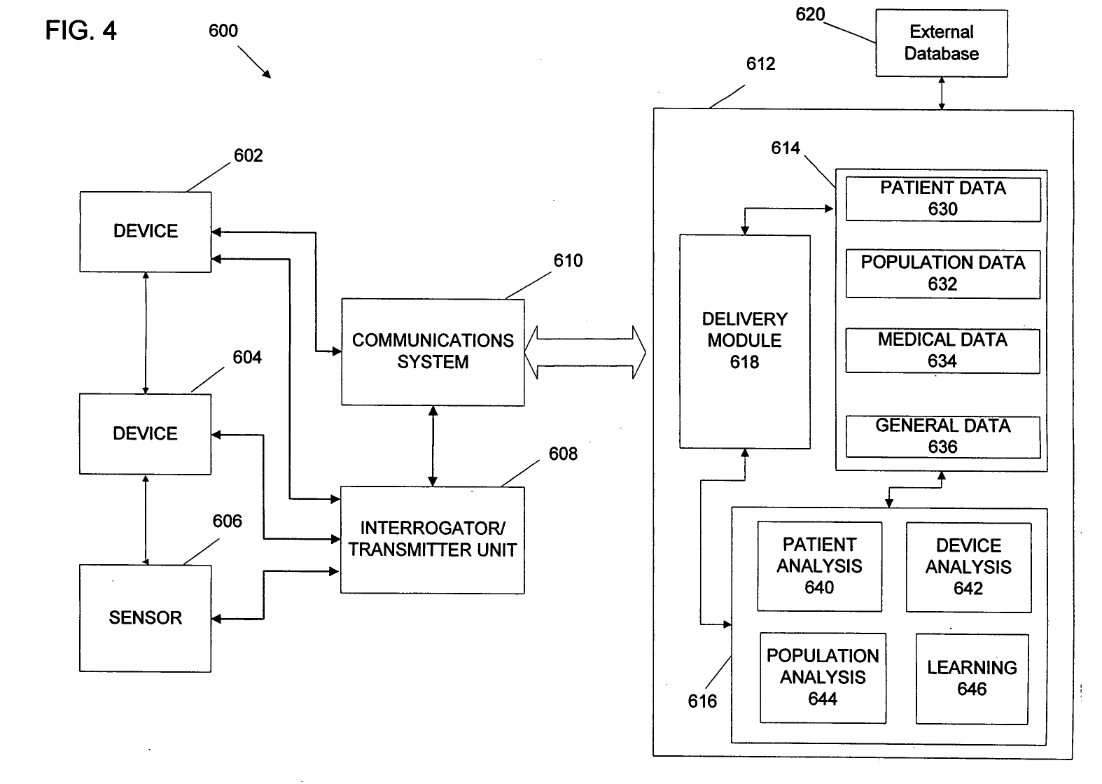

FIG. 4 illustrates an example APM system 600 made in accordance with the present invention. APM system 600 generally includes the following components: devices 602, 604, and 606, an interrogator/transceiver units 608, a communication system 610, and a host 612.

Each component of the APM system 600 can communicate using the communication system 610. Some components can also communicate directly with one another. For example, devices 602 and 604 can be configured to communicate directly with one another and with the interrogator/transceiver units 608. The various components of the example APM system 600 illustrated herein are described below.

a. Devices

Devices 602, 604, and 606 can be implantable devices or external devices that can provide at least one of the following functions with respect to a patient in addition to other possible functions: (1) sensing/measuring, (2) data analysis, (3) therapy, (4) distribution of product, and (5) communication. For example, in one embodiment, devices 602, 604, and 606 are either implanted or external devices used to sense or measure a variety of physiological, subjective, and environmental conditions of a patient using electrical, mechanical, and/or chemical means. In one embodiment, the device 602 is a handheld device, device 604 is an implanted medical device, and device 606 is an internal or external sensor.

The devices 602, 604, and 606 can be configured to automatically gather data or can require manual intervention by the patient. The devices 602, 604, and 606 can be devices that are positioned external and separated from the patient, positioned on an external surface of the patient, or positioned within the patient as an implanted device or sensor. The devices 602, 604, and 606 can be configured to store data related to the physiological and/or subjective measurements and/or transmit the data to the communication system 610 using a variety of methods, described in detail below. Although three devices 602, 604, and 606 are illustrated in the example embodiment shown, more or fewer devices can be used for a given patient.

The devices 602, 604, and 606 can be configured to analyze the measured data and act upon the analyzed data. For example, the devices 602, 604, and 606 are configured to modify therapy or provide alarm indications based on the analysis of the data.

In one embodiment, devices 602, 604, and 606 also provide therapy. Therapy can be provided automatically or in response to an external communication. Devices 602, 604, and 606 are programmable in that the characteristics of their sensing, therapy (e.g., duration and interval), or communication can be altered by communication between the devices 602, 604, and 606 and other components of the APM system 600. Devices 602, 604, and 606 can also perform self-checks or be interrogated by the communication system 610 to verify that the devices are functioning properly.

In another embodiment, devices 602, 604, and 606 also provide disbursement of product. Product disbursement can be provided automatically or in response to an external communication. Some example products that can be dispersed include pills/drugs that are part of a patient drug regimen and testing/sampling products for patient conducted tests or sampling bodily products.

The devices 602, 604, and 606 can be configured to communicate with the patient and with other devices and features of the APM. For example, the devices 602, 604, and 606 can communicate with a patient using sound or visual prompts to, for example, obtain answers to questions, remind the patient to perform certain tasks, and warn the patient about the presence of predetermined threshold trends and conditions that represent the patient's well-being. The devices 602, 604, and 606 can also include user interface features such as a keypad, touch control screen, or other input device that facilitate communication between the patient and the devices 602, 604, and 606. Additional examples of different embodiments of the devices 602, 604, and 606 are provided below.

Devices implanted within the body have the ability to sense and communicate as well as to provide therapy. Implantable devices can provide direct measurement of characteristics of the body, including, without limitation, electrical cardiac activity (e.g., a pacemaker, cardiac resynchronization management device, defibrillator, etc.), physical motion, temperature, heart rate, activity, blood pressure, breathing patterns, ejection fractions, blood viscosity, blood chemistry, blood glucose levels, and other patient-specific clinical physiological parameters, while minimizing the need for patient compliance.

Derived measurements can also be determined from the implantable device sensors. Examples of such derived measurements include, but are not limited to, a functional capacity indicator, autonomic tone indicator, sleep quality indicator, cough indicator, anxiety indicator, and cardiovascular wellness indicator for calculating a quality of life indicator quantifying a patient's overall health and well-being.

Devices 602, 604, and 606 can also be external devices, or devices that are not implanted in the human body, that are used to measure physiological data. Such devices include a multitude of devices to measure data relating to the human body, such as temperature (e.g., a thermometer), blood pressure (e.g., a sphygmomanometer), blood characteristics (e.g., glucose levels), body weight, physical strength, mental acuity, diet, heart characteristics, and relative geographic position (e.g., a Global Positioning System (GPS)). The physiologic signals collected by external sensors could be uniquely associated with the patient by verifying device ID via a telemetry link.

Devices 602, 604, and 606 can also be environmental sensors. The devices can be placed in a variety of geographic locations (in close proximity to patient or distributed throughout a population) and record nonpatient specific characteristics such as, but not limited to, temperature, air quality, humidity, carbon monoxide level, oxygen level, barometric pressure, light intensity, and sound.

One or more of the devices 602, 604, and 606 (for example, device 606) can be external devices that measure subjective or perceptive data from the patient. Subjective data is information related to a patient's feelings, perceptions, and/or opinions, as opposed to objective physiological data. For example, the “subjective” devices can measure patient responses to inquiries such as “How do you feel?” and “How is your pain?” The device can prompt the patient and record subjective data from the patient using visual and/or audible cues.

The subjective data can be collected from the patient at set times, or, alternatively, collected whenever the patient feels like providing subjective data. The subjective data can also be collected substantially contemporaneously with physiological data to provide greater insight into overall patient conditions such as dietary related conditions. The subjective device can be any device that accepts input from a patient or other concerned individual and/or provides information in a format that is recognizable to the patient.

One or more of the devices 602, 604, and 606 (e.g., device 602) can also include one or more remote peripheral devices. The remote peripheral device can include, for example and without limitation, cellular telephones, pagers, PDA devices, facsimiles, remote computers, printers, video and/or audio devices, etc. The remote peripheral device can communicate using wired or wireless technologies and can be used by the patient or caregiver to communicate with the interrogator/transmitter unit 608, communication system 610, and/or the host 612. For example, the remote peripheral device can be used by the caregiver to receive alarms from the host 612 based on data collected from the patient and to send instructions from the caregiver to either the patient or other clinical staff. In another example, the remote peripheral device is used by the patient to receive periodic or real time updates and alarms regarding the patient's health and well-being.

b. Interrogator/Transceiver Unit

Referring now to FIG. 5, the example APM system 600 includes one or more interrogator/transceiver units (“ITUs”), such as ITU 608. In illustrated embodiments, the ITU is configured in a manner similar to that disclosed in U.S. Published Patent Application No. US-2004-0127958-A1, filed Dec. 27, 2002, and entitled “Advanced Patient Management System Including Interrogator/Transceiver Unit,” which is hereby incorporated by reference in its entirety.

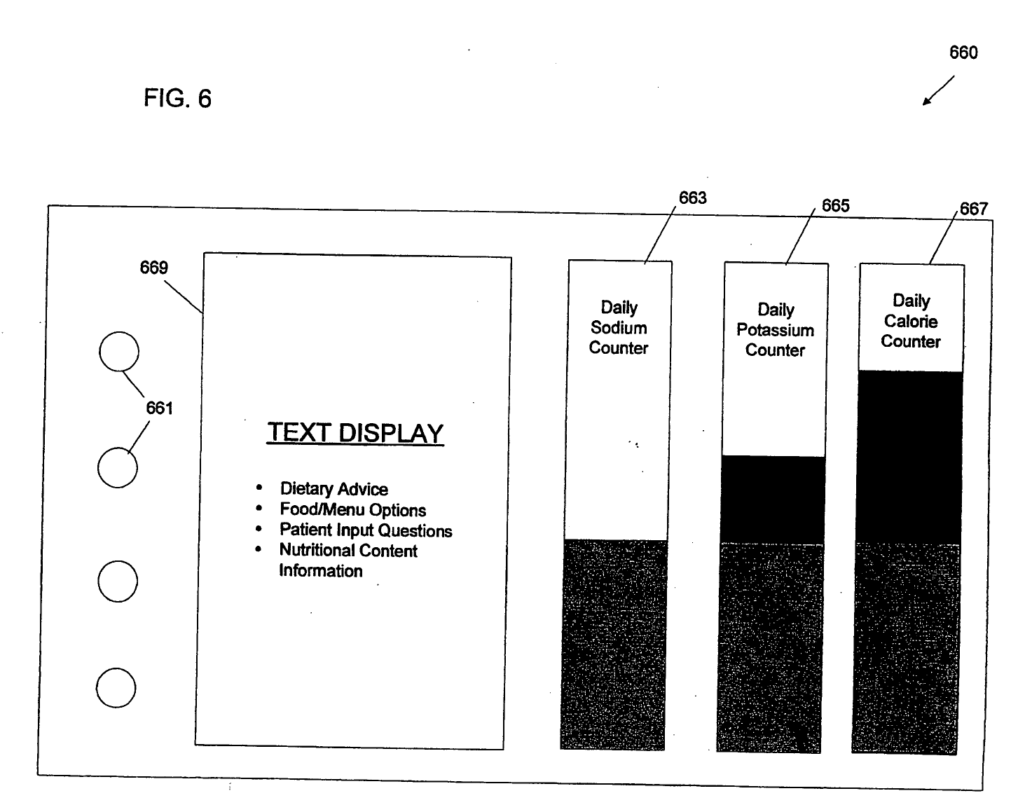

The ITU 608 can include an interrogator module 652 for sending and receiving data from a device, such as devices 602, 604, 606, a memory module 654 for storing data, and a transceiver module 656 for sending and receiving data to and from other components of the APM system 600. The transceiver module can also operate as an interrogator of the devices 602, 604, 606. The ITU 608 also includes a power module 658 that provides power and an interactive display 660. Example features of the interactive display 660 are described below with reference to FIG. 6.

The ITU 608 can perform one or more of the following functions: (1) data storage; (2) data analysis; (3) data forwarding; (4) patient interaction; (5) patient feedback; and (6) data communications. For example, the ITU 608 can facilitate communications between the devices 602, 604, 606 and the communication system 610. The ITU 608 can, periodically or in real-time, interrogate and download into memory clinically relevant patient data from the devices 602, 604, 606. This data includes, in the cardiac sensor context, for example, P and R-wave measurements, pacing, shocking events, lead impedances, pacing thresholds, battery voltage, capacitor charge times, ATR episodes with electrograms, tachycardia episodes with electrograms, histogram information, physiological conditions that represent efficacy and compliance of a drug regimen, and any other clinical information necessary to ensure patient health and proper device function. The data is sent to the ITU 608 by the devices 602, 604, 606 in real-time or periodically uploaded from buffers in the devices.

The ITU 608 can also allow patient interaction via the interactive display 660. For example, the display 660 can include a patient interface and allow the patient to input subjective data. In addition, the ITU 608 can provide feedback to the patient via the display 660 based on the data that has been analyzed or based on information communicated by the communication system 610.

In another embodiment, the ITU 608 includes a telemetry link from the devices to a network that forms the basis of a wireless LAN in the patient's home. The ITU 608 systematically uploads information from the devices 602, 604, 606 while the patient is sleeping, for example. The uploaded data is transmitted through the communication system 610 or directly to the host 612. In addition, in one embodiment the ITU 608 functions in a hybrid form, utilizing wireless communication when available and defaulting to a local wireless portal or a wired connection when the wireless communication becomes unavailable.

When the interrogator 652 uses radio frequency to communicate with the devices 602, 604, 606, the ITU 608 can be in the form of a small device that is placed in an inconspicuous place within the patient's residence. Alternatively, the ITU 608 can be implemented as part of a commonly used appliance in the patient's residence. For example, the ITU can be integrated with an alarm clock that is positioned near the patient's bed. In another embodiment, the ITU can be implemented as part of the patient's personal computer system. Other embodiments are also possible.

In another embodiment, the ITU 608 can comprise a hand-held device such as a PDA, cellular telephone, or other similar device that is in wireless communication with the devices 602, 604, 606. The hand-held device can upload the data to the communication system 610 wirelessly. Alternatively, the hand-held device can periodically be placed in a cradle or other similar device that is configured to transmit the data to the communication system 610.

If multiple devices, such as devices 602, 604, 606 and/or additional device, are provided for a given patient, each device can include its own means for communicating with the ITU 608 or communication system 610. Alternatively, a single telemetry system can be implemented as part of one of the devices, or separate from the devices, and each device 602, 604, and 606 can use this single telemetry system to communication with the ITU 608 or the communication system 610.

In yet another embodiment, the devices 602, 604, and 606 include wires or leads extending from devices 602, 604, and 606 to an area external of the patient to provide a direct physical connection. The external leads can be connected, for example, to the ITU 608 or a similar device to provide communications between the devices 602, 604, 606 and the other components of the APM system 600.

The APM system 600 can also involve a hybrid use of the ITU 608. For example, the devices 602, 604, 606 can intelligently communicate via short-range telemetry with the ITU when the patient is located within the patient's home and communicate directly with the communication system 610 or host 612 when the patient is traveling. This can be advantageous, for example, to conserve battery power when the devices are located near an ITU.

FIG. 6 illustrates an example interactive display 660 for use either independently or integrated with the ITU 608. The display 660 may include a plurality of input buttons 661, a plurality counter indicators 663, 665, 667, and a text display 669. The buttons 661 may provide for input of information or selection of options presented on the text display. The indicators 663, 665, 667 may be used to illustrate the patient's physical conditions measured by a plurality of sensors either internal or external positioned. In some embodiments, the indicators 663, 665, 667 may be integrated into the text display 669. The text display 669 may provide, for example, dietary advice, food/menu options, patient input questions, and nutritional content information.

The features of display 660 illustrated in FIG. 6 are exemplary only. Numerous combinations of buttons, displays indicators and other features may be integrated into a display for use in system 600 in ways that provide improved user convenience and transfer of information between the patient and the system 600.

c. Communication System

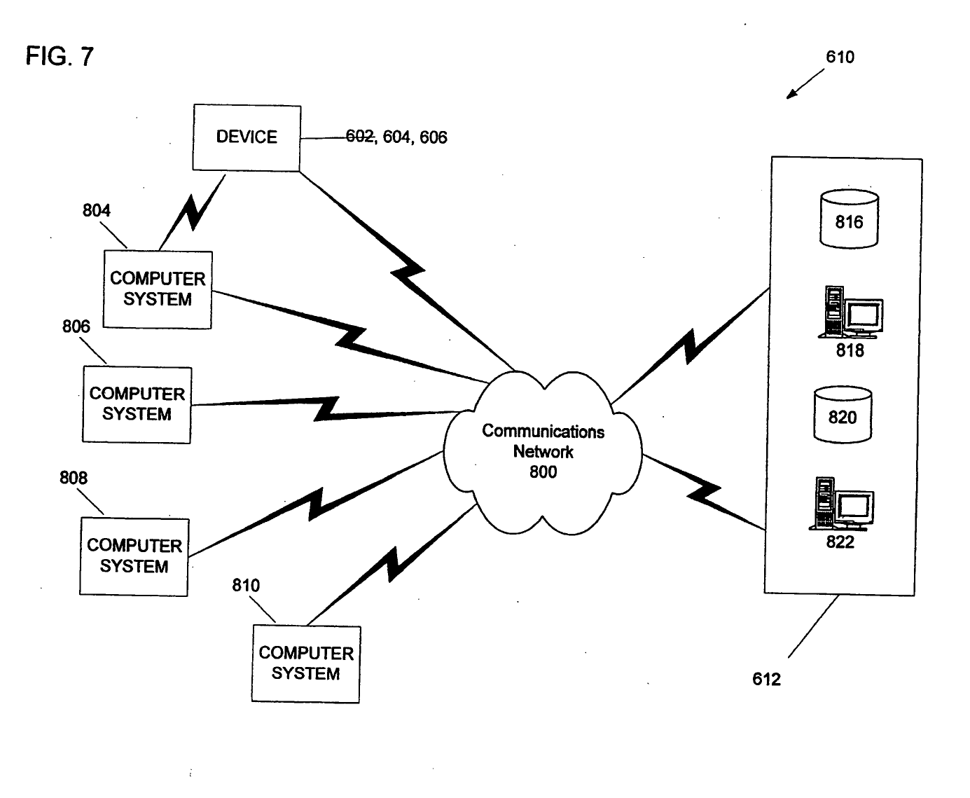

Communication system 610 provides for communications between and among the various components of the APM system 600, such as between the devices 602, 604, 606 and ITU 608 and the host 612. FIG. 7 illustrates one embodiment for the communication system 610 made in accordance with the present invention. The communication system 610 includes a plurality of computer systems 804, 806, 808, and 810, as well as devices 602, 604, 606 and host 612, connected to one another by a communication network 800. The communication network 800 can be, for example, a local area network (LAN), wide area network (WAN), or the Internet. Communications among the various components, as described more fully below, can be implemented using wired or wireless technologies.

In the example embodiment illustrated, the host 612 includes server computers 818 and 822 that communicate with computers 804, 806, 808, and 810 using a variety of communications protocols that are described more fully below. The server computers 818 and 822 store information in databases 816 and 820. This information can also be stored in a distributed manner across one or more additional servers.

A variety of communication methods and protocols can be used to facilitate communication between devices 602, 604, 606, ITU 608, communication system 610, host 612, and remote peripheral device 609. For example, wired and wireless communications methods can be used. Wired communication methods can include, for example and without limitation, traditional copper-line communications such as DSL, broadband technologies such as ISDN and cable modems, and fiber optics, while wireless communications can include cellular, satellite, radio frequency (RF), Infrared, etc.

d. Host

Referring again to FIG. 4, the example host 612 includes a database module 614, an analysis module 616, and a delivery module 618. Host 612 preferably includes enough processing power to analyze and process large amounts of data collected from each patient, as well as to process statistics and perform analysis for large populations. For example, the host 612 can include a mainframe computer or multi-processor workstation. The host 612 can also include one or more personal computer systems containing sufficient computing power and memory. The host 612 can include storage medium (e.g., hard disks, optical data storage devices, etc.) sufficient to store the massive amount of high-resolution data that is collected from the patients and analyzed.

The host 612 can also include identification and contact information (e.g., IP addresses, telephone numbers, or a product serial number) for the various devices communicating with it, such as ITU 608 and one or more of devices 602, 604, 606. For example, each ITU 608 is assigned a hard-coded or static identifier (e.g., IP address, telephone number, etc.), which allows the lost 612 to identify which patient's information the host 612 is receiving at a given instant. Alternatively, each device 602, 604, 606 can be assigned a unique identification number, or a unique patient identification number can be transmitted with each transmission of patient data.

Referring again to FIG. 4, the example database module 614 includes a patient database 630, a population database 632, a medical database 634, and a general database 636. The patient database 630 includes patient specific data, including data acquired by the devices 602, 604, and 606. The patient database 630 also includes a patient's medical records, the patient's current health information, targeted health information, and drug information. The patient database 630 can include pharmacogenomic information describing individual genetic differences that could impact drug metabolism. The patient database 630 can include historical information regarding the devices 602, 604, 606. The information stored in the database 630 can be recorded at various times depending on the patient requirements or device requirements. For example, the database 630 is updated at periodic intervals that coincide with the patient downloading data from the device. Alternatively, data in the database 630 can be updated in real time. Typically, the sampling frequency depends on the health condition being monitored and the co-morbidities.

The population database 632 includes non-patient specific data, such as data relating to other patients and population trends. The population database 632 also records epidemic-class device statistics and patient statistics. The population database 632 also includes data relating to staffing by health care providers, environmental data, drugs, etc. In some cases, patient information from the patient database 630 can be added to the population database to supplement and maintain currency of the population database information and trends.

The example medical database 634 includes clinical data relating to the treatment of diseases. For example, the medical database 634 includes historical trend data for multiple patients in the form of a record of progression of their disease(s) along with markers of key events. The medical database could also include clinical study results.

The general database 636 includes non-medical data of interest to the patient. The general database 636 can include information relating to, for example, news, finances, shopping, technology, entertainment, and/or sports. The general database 636 can be customized to provide general information of specific interest to the patient. For example, stock information can be presented along with the latest health information as detected from the devices 602, 604, and 606.

In another embodiment, information is also provided from an external source, such as external database 620. For example, the external database 620 can include external medical records related to other medical devices or medical conditions of the patient that are maintained separately and remotely from the host 612. The remote database may include standards, recommendations, or guidelines provided by medical groups and associations such as, for example, the American Hospital Association (AHA), the American Medical Association (AMA), the Heart Rhythm Society (HRS), and the Heart Failure Society of America (HFSA).

The example analysis module 616 includes a patient analysis module 640, device analysis module 642, population analysis module 644, and learning module 646. Patient analysis module 640 can utilize information collected by the APM system 600, as well as information from other relevant sources, to analyze data related to a patient and provide timely and predictive assessments of the patient's well-being. In performing this analysis, the patient device module 640 can utilize data collected from a variety of sources, include patient specific physiological and subjective data collected by the APM system 600, medical and historical records (e.g., lab test results, histories of illnesses, etc., drugs currently and previously administered, etc.), as well as information related to population trends provided from sources external to the APM system 600.

For example, in one embodiment, the patient analysis module 640 makes a predictive diagnosis of an oncoming event based on information stored in the database module 614. For example, the data continuously gathered from a device of a given patient at a heightened risk for a chronic disease event (such as de-compensations in heart failure) is analyzed. Based on this analysis, therapy, typically device-based, dietary, or drug, can then be applied to the patient either through the device, via communications to the patient, or through clinician intervention.

In another example embodiment, the patient analysis module 640 provides a diagnosis of patient health status and predicted trend based on present and recent historical data collected from a device as interpreted by a system of expert knowledge derived from working practices within clinics. For example, the patient analysis module 640 performs probabilistic calculations using currently-collected information combined with regularly-collected historical information to predict patient health degradation.

In another example embodiment, the patient analysis module 640 can conduct pre-evaluation of the incoming data stream combined with patient historical information and information from patients with similar disease states. The pre-evaluation system is based on data derived from working clinical practices and the records of outcomes. The derived data is processed in an expert system (i.e., neural network, fuzzy logic system, or equivalent system) to reflect the clinical practice. Further, the patient analysis module 640 can also provide means for periodic processing of present and historical data to yield a multidimensional health state indication along with disease trend prediction, next phase of disease progression co- morbidities, and inferences about what other possible diseases can be involved. The patient analysis module 640 can also integrate data collected from internal and external devices with subjective data to optimize management of overall patient health.

Device analysis module 642 analyzes data from the devices 602, 604, 606 and ITU 608 to predict and determine device issues or failures. For example, if an implanted device 602 fails to communicate at an expected time, device analysis module 642 determines the source of the failure and takes action to restore the performance of the device 602. The device analysis module 642 can also perform additional deterministic and probabilistic calculations. For example, the device analysis module 642 gathers data related to charge levels within a given device, such as an ICD, and provides analysis and alarming functions based on this information if, for example, the charge level reaches a point at which replacement of the device and/or battery is necessary. Similarly, early degradation or imminent failure of implanted devices or leads can be identified and proactively addressed, or at-risk devices can be closely monitored.

Population analysis module 644 uses the data collected in the database module 614 to manage the health of a population. For example, a clinic managing cardiac patients can access the APM system 600 and thereby obtain device-supplied advance information to predict and optimize resource allocation both as to immediate care and as a predictive metric for future need of practicing specialists. As another example, the spread of disease in remote populations can be localized and quarantined rapidly before further spread.

In one embodiment, population analysis module 644 trends the patient population therapy and management as recorded by the devices and directs health care resources to best satisfy the needs of the population. The resources can include people, facilities, supplies, and/or drugs. In other embodiments, the population analysis module detects epidemics and other events that affect large population groups. The population analysis module 644 can issue alarms that can initiate a population quarantine, redirect resources to balance size of staffing with number of presenting population, and predict future need of qualified specialists.

The population analysis module 644 can utilize a variety of characteristics to identify like-situated patients, such as, for example, sex, age, genetic makeup, etc. The population analysis module 644 can develop large amounts of data related to a given population based on the information collected by the APM system 600. In addition, the population analysis module 644 can integrate information from a variety of other sources. For example, the population analysis module 644 can utilize data from public domain databases (e.g., the National Institute of Health), public and governmental and health agency databases, private insurance companies, medical societies (e.g., the American Heart Association), and genomic records (e.g., DNA sequences).

In one embodiment of the invention, the host 612 can be used as a “data clearinghouse,” to gather and integrate data collected from the devices 602, 604, 606, as well as data from sources outside the APM system 600, such as the external database 600. The integrated data can be shared with other interested entities, subject to privacy restrictions, thereby increasing the quality and integration of data available.

Learning module 646 analyzes the data provided from the various information sources, including the data collected by the advanced patient system 600 and external information sources. For example, the learning module 646 analyzes historical symptoms, diagnoses, and outcomes along with time development of the diseases and co-morbidities. The learning module 646 can be implemented via an expert system.

The learning module 646 can be partially trained (i.e., the learning module 646 can be implemented with a given set of inference rules and then learn as the APM system functions) or untrained (i.e., the learning module 646 is initiated with no preset values and must learn from scratch as the APM system functions). In other alternative embodiments, the learning module 646 can continue to learn and adjust as the APM system functions (i.e., in real time), or the learning module 646 can remain at a given level of learning and only advanced to a higher level of understanding when manually allowed to do so.

In an expert system embodiment, new clinical information is presented to create new neural network coefficients that are distributed as an expert system knowledge upgrade. The learning module 646 can include a module for verifying the expert system conclusions for clinical accuracy and significance. The learning module can analyze a database of test cases, appropriate outcomes and relative occurrence of misidentification of the proper outcomes. In some embodiments, the learning module 646 can update the analysis module 616 when the analysis algorithms exceed a threshold level of acceptable misidentifications.

The example learning module 646 uses various algorithms and mathematical modeling such as, for example, trend and statistical analysis, data mining, pattern recognition, cluster analysis, neural networks and fuzzy logic. Learning module 646 can perform deterministic and probabilistic calculations. Deterministic calculations include algorithms for which a clear correlation is known between the data analyzed and a given outcome. For example, there can be a clear correlation between the energy left in a battery of an implantable device and the amount of time left before the battery must be replaced.

A probabilistic calculation involves the correlation between data and a given outcome that is less than 100 percent certain. Probabilistic determinations require an analysis of several possible outcomes and an assignment of probabilities for those outcomes (e.g., an increase in weight of a patient can, at a 25 percent probability, signal an impending de-compensation event and/or indicate that other tests are needed). The learning module 646 performs probabilistic calculations and selects a given response based on a highest probability. In doing so the module could use prior probability of an event derived from population or clinical study database. Further, as the learning module 646 “learns” for previous determinations (e.g., through a neural network configuration), the learning module 646 becomes more proficient at assigning probabilities for a given data pattern, thereby being able to more confidently select a given response. As the amount of data that has been analyzed by the learning module 646 grows, the learning module 646 becomes more and more accurate at assigning probabilities based on data patterns. A bifurcated analysis can be performed for diseases exhibiting similar symptoms. As progressive quantities of data are collected and the understanding of a given disease state advances, disease analysis is refined where a former singular classification can split into two or more sub-classes.

In addition, patient-specific clinical information can be stored and tracked for hundreds of thousands of individual patients, enabling a first- level electronic clinical analysis of the patient's clinical status and an intelligent estimate of the patient's short-term clinical prognosis. The learning module 646 is capable of tracking and forecasting a patient's clinical status with increasing levels of sophistication by measuring a number of interacting co-morbidities, all of which can serve individually or collectively to degrade the patient's health. This enables learning module 646, as well as caregivers, to formulate a predictive medical response to oncoming acute events in the treatment of patients with chronic diseases such as heart failure, diabetes, renal dysfunction, cancer, and asthma/COPD, as well as possibly head-off acute catastrophic conditions such as MI and stroke.

Delivery module 618 coordinates the delivery of feedback based on the analysis performed by the host 612. In response to the analysis module 616, delivery module 618 can manage the devices 602, 604, 606, perform diagnostic data recovery, program the devices, and otherwise deliver information as needed. In some embodiments, the delivery module 618 can manage a web interface that can be accessed by patients or caregivers. The information gathered by an implanted device or sensor, external sensor, or handheld device associated with system 600 can be periodically transmitted to a web site that is securely accessible to the caregiver and/or patient in a timely manner. In other embodiments, a patient accesses detailed health information with diagnostic recommendations based upon analysis algorithms derived from leading health care institutions.

For example, the caregiver and/or patient can access the data and analysis performed on the data by accessing one or more general content providers. In one example, the patient's health information is accessed through a general portal such as My Yahoo provided by Yahoo! Inc. of Sunnyvale, California, or Guidant patient personal web page provided by Guidant Corporation of Indianapolis, Indiana. For example, a patient can access his or her My Yahoo homepage or Guidant patient personal web page and receive information regarding current health and trends derived from the information gathered from the devices 602, 604, and 606, as well as other health information gathered from other sources. The patient can also access other information in addition to health information on the My Yahoo website, such as weather and stock market information. Other electronic delivery methods such as email, facsimile, etc. can also be used for alarm distribution.

In an alternative embodiment, the data collected and integrated by the advanced patient system 600, as well as any analysis performed by the system 600, is delivered by delivery module 618 to a caregiver's hospital computer system for access by the caregiver. A standard or custom interface facilitates communication between the APM system 600 and a legacy hospital system used by the caregiver so that the caregiver can access all relevant information using a system familiar to the caregiver.

The APM system 600 can also be configured so that various components of the system (e.g., ITU 608, communication system 610, and/or host 612) provide reporting to various individuals (e.g., patient and/or caregiver). For example, different levels of reporting can be provided by (1) the ITU 608 and (2) the host 612. The ITU 608 can be configured to conduct rudimentary analysis of data gathered from devices 602, 604, 606, and provide reporting should an acute situation be identified. For example, if the ITU 608 detects a significantly high or low level of certain nutritional parameters that relate to a patient's immediate wellbeing, the ITU 608 provides reporting to the patient in the form of an audible or visual alarm.

The host 612 can provide a more sophisticated reporting system. For example, the host 612 can provide exception-based reporting and alarms that categorize different reporting events based on importance. Some reporting events do not require caregiver intervention and therefore can be reported automatically. In other escalating situations, caregiver and/or emergency response personnel need to become involved. For example, based on the data collected by the APM system 600, the delivery module 618 can communicate directly with the devices 602, 604, 606, communicate with the patient via a display (e.g., an interactive display on the ITU 608), and/or contact 911 emergency response. In an alternative embodiment, the delivery module 618 and/or the patient can also establish a voice communication link between the patient and a caregiver, if warranted.

In addition to forms of reporting including visual and/or audible information, the APM system 600 can also communicate with and reconfigure one or more of the devices 602, 604, 606. For example, if device 602 is part of a cardiac rhythm management system, the host 612 can communicate with the device 602 and reconfigure the therapy provided by the cardiac rhythm management system based on the data collected from one or more of the devices 602, 604, 606. In another embodiment, the delivery module 618 can provide to the ITU 608 recorded data, an ideal range for the data, a conclusion based on the recorded data, and a recommended course of action. This information can be displayed on the ITU 608 for the patient to review or made available on the peripheral device 609 for the patient and/or clinician to review.

III. Example Methods of Using the Advanced Patient Management System

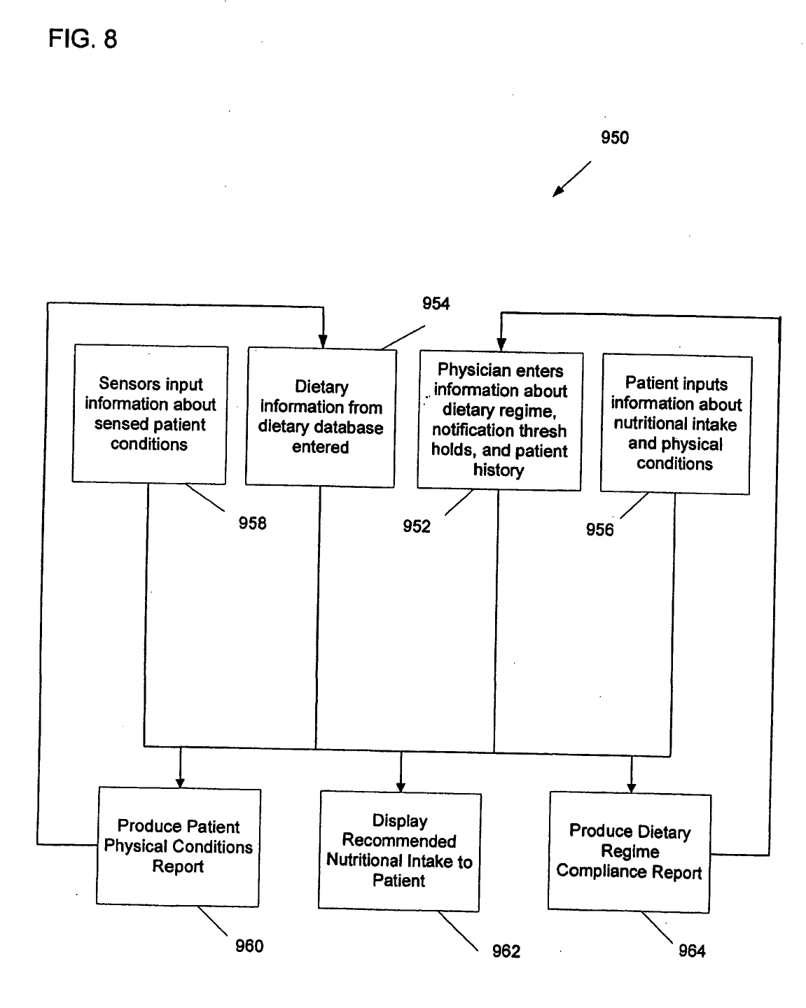

A method 950 of using an APM system according to principles of the present invention is further illustrated with reference to FIG. 8. The method includes input of different types of information from different sources and output of information related to a patient's dietary conditions. The method includes a step 952 of the physician entering information about a suggested dietary regimen (e.g., types of food/nutritional intake, interval, and amount), notification thresholds, and the patient health history into the APM system. Another step 954 includes entering of dietary information from dietary databases. Dietary databases may include, for example, nutritional and side-effects information about different types of food, relationships between foods and different types of activities, etc. A further step 956 includes patient inputs about nutritional intake and physical conditions. Another step 958 includes input from sensors related to measured patient conditions.

One or more outputs 960, 962, 964 result from one or more system inputs. One example output 960 includes a patient physical conditions report. Another example output 962 includes a displayed recommendation to the patient of nutritional intake and/or physical activity. A further example output 964 includes a compliance report related to a patient's adherence to a recommended nutritional intake schedule.

Another example method (not illustrated) may include a scenario generator. The system could provide for patient inputs that relate to a proposed nutritional intake and/or activity schedule for a given day. The system would then output estimated dietary and other health related conditions that would likely result if the patient actually undertook the proposed schedule. This output could provide valuable insight to the patient when making decisions concerning the patient's menu and activity selection. These and other methods could result from applying principles of the example disclosed above to meet other objectives of the patient or patient caregiver.

IV. Conclusion

One or more headings have been provided above to assist in describing the various embodiments disclosed herein. The use of headings, and the resulting division of the description by the headings, should not be construed as limiting in any way. The subject matter described under one heading can be combined with subject matter described under one or more of the other headings without limitation and as desired.

The systems and methods of the present disclosure can be implemented using a system as shown in the various Figures disclosed herein including various devices and/or programmers, including implantable or external devices. Accordingly, the methods of the present disclosure can be implemented: (1) as a sequence of computer implemented steps running on the system; and (2) as interconnected modules within the system. The implementation is a matter of choice dependent on the performance requirements of the system implementing the method of the present disclosure and the components selected by or utilized by the users of the method.

Accordingly, the logical operations making up the embodiments of the method of the present disclosure described herein can be referred to variously as operations, steps, or modules. It will be recognized by one of ordinary skill in the art that the operations, steps, and modules can be implemented in software, in firmware, in special purpose digital logic, analog circuits, and any combination thereof without deviating from the spirit and scope of the present invention as recited within the claims attached hereto.

The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. An advanced patient management system, comprising:
a device configured to assess at least one dietary condition of a patient;
an interactive display configured to receive patient input related to the patient's dietary conditions; and
a repeater unit configured to collect information from the device and the interactive display;
wherein the system provides dietary related feedback to the patient in response to the collected information.

2. The system of claim 1, wherein the system provides dietary related feedback to the patient via the interactive display.

3. The system of claim 1, further comprising at least one sensor configured to assess at least one dietary condition of the patient, and the repeater unit is configured to collect information from the at least one sensor.

4. The system of claim 3, wherein the at least one sensor is internally implanted in the patient.

5. The system of claim 3, wherein the at least one sensor is positioned external of the patient.

6. The system of claim 3, wherein the at least one sensor is selected from a group comprising a sodium sensor, a weight measuring sensor, a blood pressure sensor, a heart rate sensor, an INR/Coumadin sensor, a glucose sensor, a respiration sensor, an insulin sensor, a temperature sensor, and a hydration sensor.

7. The system of claim 1, further comprising a host in communication with the repeater through a network.

8. The system of claim 7, wherein the host is configured to receive physician input related to dietary conditions of the patient.

9. The system of claim 7, wherein the host is configured to receive information from remote databases.

10. The system of claim 7, wherein the host stores and communicates support algorithms to the repeater.

11. The system of claim 1, wherein the repeater includes the interactive display.

12. The system of claim 1, wherein the device is an implanted medical device selected from a group comprising a cardiac rhythm management (CRM) device, a cardiac diagnostic device, a loop recorder, and sodium, glucose, temperature, electrolyte, hydration, respiratory, heart rate, and cardiac output sensors.

13. The system of claim 1, further comprising a handheld unit, the handheld unit being configured to communicate dietary information between the device and the handheld unit and between the repeater and the handheld unit.

14. The system of claim 2, wherein the dietary feedback includes at least one of dietary advice, food and menu options, calorie information, sodium information, hydration conditions, pharmacological dosage recommendations, and patient input prompts.

15. A method of monitoring dietary conditions of a patient, the method including:
collecting dietary information from the patient with an implanted device;
comparing the collected dietary information to expected dietary conditions of the patient stored by a repeater or a host; and
communicating dietary information to the patient in response to the comparison.

16. The method of claim 15, further comprising collecting dietary information from the patient via an interactive display.

17. The method of claim 16, further comprising measuring a dietary condition of the patient with at least one sensor, and comparing the dietary condition to expected dietary conditions of the patient stored by the repeater or the host.

18. The method of claim 17, wherein the dietary information collected via the interactive display is used to confirm the dietary condition measured by the at least one sensor and the dietary information collected by the implanted device.

19. The method of claim 16, wherein communicating dietary information to the patient includes displaying via the interactive display at least one of a suggested nutritional intake menu, dietary advice, calorie information, sodium information, hydration conditions, and pharmacological dosage recommendations.

20. An advanced patient management system, comprising:
a repeater unit;
an implanted medical device configured to communicate physiological information about a patient to the repeater unit;
at least one implanted or external sensor configured to measure at least one dietary condition of the patient and communicate the measured condition to the repeater unit;
an interactive display configured to receive dietary information input from the patient and to display information to the patient; and
a host configured to communicate with the repeater via a communications network and to store information related to the patient.

21. The system of claim 20, wherein the at least one sensor is selected from a group comprising a sodium sensor, a weight measuring sensor, a blood pressure sensor, a heart rate sensor, an INR/Coumadin sensor, a glucose sensor, a respiration sensor, an insulin sensor, a temperature sensor, and a hydration sensor.

22. The system of claim 20, wherein the device is an implanted medical device selected from a group comprising a cardiac rhythm management (CRM) device, a cardiac diagnostic device, a loop recorder, and sodium, glucose, temperature, electrolyte, hydration, respiratory, heart rate, and cardiac output sensors.

23. A method of monitoring and communicating a patient's nutritional/dietary condition, the method comprising:
collecting data associated with at least one dietary condition of a patient;
communicating the data to a host;
comparing the collected data to an expected dietary model; and
communicating to the patient a recommended nutritional intake.

24. The method of claim 23, wherein communicating to the patient includes displaying a list of types and amounts of food.

25. The method of claim 24, wherein the comparing step further comprises determining dietary regimen compliance based on comparison of the data to the expected dietary model.
